(19) United States
(12) Patent Application Publication
(10) Pub. No.: US 2024/0382601 A1
(43) Pub. Date: Nov. 21, 2024

(54) EMULSION FORMULATION HAVING HIGH FORMULATION STABILITY AND MANUFACTURING METHOD THEREOF

(71) Applicants: AMOREPACIFIC CORPORATION, Seoul (KR); DKBIO CO., LTD, Daejeon (KR)

(72) Inventors: Jaewon YOU, Yongin-si (KR); Suil KIM, Yongin-si (KR); Yujung KIM, Yongin-si (KR); Jong Eoun HONG, Seoul (KR)

(73) Assignees: AMOREPACIFIC CORPORATION, Seoul (KR); DKBIO CO., LTD, Daejeon (KR)

(21) Appl. No.: 18/660,430

(22) Filed: May 10, 2024

(30) Foreign Application Priority Data
May 15, 2023 (KR) 10-2023-0062299

(51) Int. Cl. A61K 47/54; A61K 9/107

(52) U.S. Cl. CPC A61K 47/549; A61K 9/107; A61K 47/54

(57) ABSTRACT

In one aspect, the present disclosure provides a complex containing kojyl methylenedioxycinnamate; a compound containing an ethylene glycol moiety or a propylene glycol moiety; a surfactant containing a saccharide moiety; and an antioxidant, and an emulsion formulation containing the complex. In another aspect, the present disclosure provides a method for manufacturing the complex, comprising: (a) adding kojyl methylenedioxycinnamate, a compound containing an ethylene glycol moiety or a propylene glycol moiety, a surfactant containing a saccharide moiety, and an antioxidant; and (b) homomixing the result of the (a).

### CROSS-REFERENCE TO RELATED APPLICATION

The present application claims priority to Korean Patent Application No. 10-2023-0062299, filed May 15, 2023, the entire content of which is incorporated herein by this reference.

### BACKGROUND OF THE INVENTION

### Field of the Invention

The present disclosure relates to an emulsion formulation with high formulation stability containing kojyl methylenedioxycinnamate and a method for manufacturing the same.

### Description of the Related Art

Kojyl methylenedioxycinnamate (brand name: Seletinoid G, Seletinoid G™) is known to have particularly excellent anti-aging, antioxidant, and whitening effects, but its strong crystallinity makes it difficult to dissolve in the composition. In addition, even after dissolution, recrystallization or precipitation occurs quickly, reducing the product's skin absorption rate or feeling of use. Because of these problems, the use of kojyl methylenedioxycinnamate as an active ingredient in the cosmetic composition was realistically limited despite its excellent efficacy.

Various attempts have been made to solve this problem of poor solubility, but since it is essential to dissolve kosyl methylenedioxycinnamate at a high temperature for a long time, there is a high possibility that the content of kosyl methylenedioxycinnamate will decrease during this process.

Accordingly, there is a need for research into increasing the stability of the formulation while maintaining the content of kojyl methylenedioxycinnamate.

### SUMMARY OF THE INVENTION

In one aspect, the object is to provide a complex comprising kojyl methylenedioxycinnamate.

In one aspect, the object is to provide an emulsion formulation comprising the complex.

In one aspect, the kojyl methylenedioxycinnamate is included in the complex to stabilize it without precipitating within the formulation.

In one aspect, the content of kojyl methylenedioxycinnamate is maintained in the complex and the emulsion formulation.

In one aspect, the object is to provide a method for manufacturing the complex.

In one aspect, the object is to provide a method for manufacturing an emulsion formulation comprising the complex.

In order to achieve the above object, in one aspect, the present invention provides a complex comprising kojyl methylenedioxycinnamate; a compound comprising an ethylene glycol moiety or a propylene glycol moiety; a surfactant comprising a saccharide moiety; and an antioxidant.

In another aspect, the present invention provides an emulsion formulation comprising the complex.

In another aspect, the present invention provides a composition for stabilizing the kojyl methylenedioxycinnamate, wherein the composition comprises a compound comprising an ethylene glycol moiety or a propylene glycol moiety; a surfactant comprising a saccharide moiety; and an antioxidant as an active ingredient, and forms a complex with kojyl methylenedioxycinnamate to stabilize the kojyl methylenedioxycinnamate in an emulsion formulation.

In another aspect, the present invention provides a method for manufacturing the complex, comprising: (a) adding kojyl methylenedioxycinnamate, a compound comprising an ethylene glycol moiety or a propylene glycol moiety, a surfactant comprising a saccharide moiety, and an antioxidant; and (b) homomixing the result of the (a).

In another aspect, the present invention provides a method for manufacturing the emulsion formulation, comprising: (a) preparing an emulsion by preparing an aqueous phase and an oil phase; (b) independently of the (a), adding kojyl methylenedioxycinnamate, a compound comprising an ethylene glycol moiety or a propylene glycol moiety, a surfactant comprising a saccharide moiety, and an antioxidant; (c) homomixing the result of the (b); and (d) adding the result of the (c) to the oil phase of the (a).

### Advantageous Effects of Invention

In one aspect, the complex according to an embodiment of the present invention can be stabilized while containing kojyl methylenedioxycinnamate,

In one aspect, the emulsion formulation according to an embodiment of the present invention can secure a stable formulation while containing kojyl methylenedioxycinnamate,

In one aspect, the complex or emulsion formulation according to an embodiment of the present invention maintains the content of kojyl methylenedioxycinnamate and increases skin absorption rate.

### BRIEF DESCRIPTION OF THE DRAWINGS

FIG. 1 is a graph confirming the change in content of kojyl methylenedioxycinnamate in an emulsion formulation over time.

FIG. 2 is a graph confirming the degree of skin absorption of kojyl methylenedioxycinnamate in an emulsion formulation.

### DETAILED DESCRIPTION OF THE INVENTION

Hereinafter, the present invention will be described in detail.

The present invention, in one aspect, provides a complex comprising kojyl methylenedioxycinnamate; a compound comprising an ethylene glycol moiety or a propylene glycol moiety; a surfactant comprising a saccharide moiety; and an antioxidant.

In one embodiment, the kojyl methylenedioxycinnamate may be contained in an amount of 0.1 to 5% by weight based on the total weight of the complex. For example, the kojyl methylenedioxycinnamate may be contained in an amount of 0.1% by weight or more, 0.2% by weight or more, 0.3% by weight or more, 0.5% by weight or more, 0.6% by weight or more, 0.7% by weight or more, 0.8% by weight or more, 0.9% by weight or more, 1% by weight or more, 1.1% by weight or more, 1.2% by weight or more, 1.3% by weight or more, 1.4% by weight or more, 1.5% by weight or more, 1.6% by weight or more, 5% by weight or less, 4.5% by weight or less, 4% by weight or less, 3.5% by weight or less, 3% by weight or less, 2.5% by weight or less, 2.4% by weight or less, 2.3% by weight or less, 2.2% by weight or less, 2.1% by weight or less, 2% by weight or less, 1.9% by weight or less, 1.8% by weight or less, or 1.7% by weight or less.

In one embodiment, the compound comprising an ethylene glycol moiety or a propylene glycol moiety may comprise two or more ethylene glycol moieties or propylene glycol moieties.

In one embodiment, the compound comprising an ethylene glycol moiety or a propylene glycol moiety may be contained in an amount of 20 to 60% by weight based on the total weight of the complex. For example, the compound comprising an ethylene glycol moiety or a propylene glycol moiety may be 20% by weight or more, 21% by weight or more, 22% by weight or more, 23% by weight or more, 24% by weight or more, 25% by weight or more, 26% by weight or more, 27% by weight or more, 28% by weight or more, 29% by weight or more, 30% by weight or more, 31% by weight or more, 32% by weight or more, 33% by weight or more, 34% by weight or more, 35% by weight or more, 36% by weight or more, 37% by weight or more, 38% by weight or more, 39% by weight or more, 40% by weight or more, 41% by weight or more, 42% by weight or more, 43% by weight or more, 44% by weight or more, 45% by weight or more, 60% by weight or less, 59% by weight or less, 58% by weight or less, 57% by weight or less, 56% by weight or less, 55% by weight or less, 54% by weight or less, 53% by weight or less, 52% by weight or less, 51% by weight or less, 50% by weight or less, 49% by weight or less, 48% by weight or less, 47% by weight or less, or 46% by weight or less.

In one embodiment, the compound comprising an ethylene glycol moiety or a propylene glycol moiety may have a ratio of carbon and oxygen atoms in the compound of 1.5 to 3:1. For example, based on the number of oxygen atoms in the compound as 1, the number of carbon atoms may be 1.5 or more, 1.6 or more, 1.7 or more, 1.8 or more, 1.9 or more, 2 or more, 2.1 or more, 2.2 or more, 2.25 or more, 3 or less, 2.9 or less, 2.8 or less, 2.7 or less, 2.6 or less, 2.5 or less, 2.4 or less, or 2.3 or less.

In one embodiment, the compound comprising an ethylene glycol moiety or a propylene glycol moiety may have a distribution coefficient of −2 to 2.

The distribution coefficient may be expressed by the following formula, and refers to the ratio of the concentration of a specific ingredient in the aqueous phase and the oil phase when the concentration of the specific ingredient in the two liquid phases is in equilibrium.

$${D}_{A} = \frac{{[ A ]}_{oil phase}}{{[ A ]}_{aqueous phase}}$$

Herein, D_{A} means the distribution coefficient, and [A] means the concentration of the ingredient present in a specific solvent. If the value is 1, it means that the specific ingredient has the same affinity for two different liquid phases.

For example, the distribution coefficient may be −2 or higher, '11.9 or higher, −1.8 or higher, −1.7 or higher, −1.6 or higher, −1.5 or higher, −1.4 or higher, −1.3 or higher, −1.2 or higher, −1.1 or higher, −1 or higher, −0.9 or higher, −0.8 or higher, −0.7 or higher, −0.6 or higher, −0.5 or higher, −0.4 or higher, −0.3 or higher, −0.2 or higher, −0.1 or higher, 0 or higher, 2 or lower, 1.9 or lower, 1.8 or lower, 1.7 or lower, 1.6 or lower, 1.5 or lower, 1.4 or lower, 1.3 or lower, 1.2 or lower, 1.1 or lower, 1 or lower, 0.9 or lower, 0.8 or lower, 0.7 or lower, 0.6 or lower, 0.5 or lower, 0.4 or lower, 0.3 or lower, 0.2 or lower, or 0.1 or lower.

In one embodiment, the compound comprising an ethylene glycol moiety or a propylene glycol moiety may be at least one selected from the group consisting of dipropylene glycol, diethoxyethyl succinate and polyethylene glycol 400 (PEG400).

In one embodiment, the surfactant comprising a saccharide moiety may be contained in an amount of 20 to 80% by weight based on the total weight of the complex. For example, the surfactant may be present in an amount of 20% by weight or more, 21% by weight or more, 22% by weight or more, 23% by weight or more, 24% by weight or more, 25% by weight or more, 26% by weight or more, 27% by weight or more, 28% by weight or more, 29% by weight or more, 30% by weight or more, 31% by weight or more, 32% by weight or more, 33% by weight or more, 34% by weight or more, 35% by weight or more, 36% by weight or more, 37% by weight or more, 38% by weight or more, 39% by weight or more, 40% by weight or more, 41% by weight or more, 42% by weight or more, 43% by weight or more, 44% by weight or more, 45% by weight or more, 80% by weight or less, 79% by weight or less, 78% by weight or less, 77% by weight or less, 76% by weight or less, 75% by weight or less, 74% by weight or less, 73% by weight or less, 72% by weight or less, 71% by weight or less, 70% by weight or less, 69% by weight or less, 68% by weight or less, 67% by weight or less, 66% by weight or less, 65% by weight or less, 64% by weight or less, 63% by weight or less, 62% by weight or less, 61% by weight or less, 60% by weight or less, 59% by weight or less, 58% by weight or less, 57% by weight or less, 56% by weight or less, 55% by weight or less, 54% by weight or less, 53% by weight or less, 52% by weight or less, 51% by weight or less, 50% by weight or less, 49% by weight or less, 48% by weight or less, 47% by weight or less, or 46% by weight or less.

In one embodiment, the surfactant comprising a saccharide moiety may have an HLB (Hydrophile-Lipophile Balance) value of 15.5 to 18.5. For example, the HLB value may be 15.5 or higher, 15.6 or higher, 15.7 or higher, 15.8 or higher, 15.9 or higher, 16 or higher, 16.1 or higher, 16.2 or higher, 16.3 or higher, 16.4 or higher, 16.5 or higher, 16.6 or higher, 16.7 or higher, 16.8 or higher, 16.9 or higher, 17 or higher, 18.5 or lower, 18.4 or lower, 18.3 or lower, 18.2 or lower, 18.1 or lower, 18 or lower, 17.9 or lower, 17.8 or lower, 17.7 or lower, 17.6 or lower, 17.5 or lower, 17.4 or lower, 17.3 or lower, 17.2 or lower, or 17.1 or lower.

In one embodiment, the surfactant comprising a saccharide moiety may be at least one selected from the group consisting of polysorbate 20 and sucrose laurate.

In one embodiment, the antioxidant may be contained in an amount of 0.1 to 5% by weight based on the total weight of the complex. For example, the antioxidant may be present in an amount of 0.1% by weight or more, 0.2% by weight or more, 0.3% by weight or more, 0.5% by weight or more, 0.6% by weight or more, 0.7% by weight or more, 0.8% by weight or more, 0.9% by weight or more, 1% by weight or more, 1.1% by weight or more, 1.2% by weight or more, 1.3% by weight or more, 1.4% by weight or more, 1.5% by weight or more, 1.6% by weight or more, 5% by weight or less, 4.5% by weight or less, 4% by weight or less, 3.5% by weight or less, 3% by weight or less, 2.5% by weight or less, 2.4% by weight or less, 2.3% by weight or less, 2.2% by weight or less, 2.1% by weight or less, 2% by weight or less, 1.9% by weight or less, 1.8% by weight or less, or 1.7% by weight or less.

In one embodiment, the antioxidant may be at least one selected from the group consisting of tocopherol and ferulic acid.

In another aspect, the present invention provides an emulsion formulation comprising the complex.

In one embodiment, the complex may be contained in amount of 1 to 10% by weight based on the total weight of the emulsion formulation. For example, the complex may be present in an amount of 1% by weight or more, 1.1% by weight or more, 1.2% by weight or more, 1.3% by weight or more, 1.4% by weight or more, 1.5% by weight or more, 1.6% by weight or more, 1.7% by weight or more, 1.8% by weight or more, 1.9% by weight or more, 2% by weight or more, 2.1% by weight or more, 2.2% by weight or more, 2.3% by weight or more, 2.4% by weight or more, 2.5% by weight or more, 2.6% by weight or more, 2.7% by weight or more, 2.8% by weight or more, 2.9% by weight or more, 3% by weight or more, 3.1% by weight or more, 3.2% by weight or more, 3.3% by weight or more, 3.4% by weight or more, 3.5% by weight or more, 3.6% by weight or more, 3.7% by weight or more, 3.8% by weight or more, 3.9% by weight or more, 4% by weight or more, 4.1% by weight or more, 4.2% by weight or more, 4.3% by weight or more, 4.4% by weight or more, 4.5% by weight or more, 4.6% by weight or more, 4.7% by weight or more, 4.8% by weight or more, 4.9% by weight or more, 5% by weight or more, 10% by weight or less, 9.8% by weight or less, 9.7% by weight or less, 9.6% by weight or less, 9.5% by weight or less, 9.4% by weight or less, 9.3% by weight or less, 9.2% by weight or less, 9.1% by weight or less, 9% by weight or less, 8.9% by weight or less, 8.8% by weight or less, 8.7% by weight or less, 8.6% by weight or less, 8.5% by weight or less, 8.4% by weight or less, 8.3% by weight or less, 8.2% by weight or less, 8.1% by weight or less, 8% by weight or less, 7.9% by weight or less, 7.8% by weight or less, 7.7% by weight or less, 7.6% by weight or less, 7.5% by weight or less, 7.4% by weight or less, 7.3% by weight or less, 7.2% by weight or less, 7.1% by weight or less, 7% by weight or less, 6.9% by weight or less, 6.8% by weight or less, 6.7% by weight or less, 6.6% by weight or less, 6.5% by weight or less, 6.4% by weight or less, 6.3% by weight or less, 6.2% by weight or less, 6.1% by weight or less, 6% by weight or less, 5.9% by weight or less, 5.8% by weight or less, 5.7% by weight or less, 5.6% by weight or less, 5.5% by weight or less, 5.4% by weight or less, 5.3% by weight or less, 5.2% by weight or less, or 5.1% by weight or less.

In one embodiment, the emulsion formulation may comprise an aqueous phase and an oil phase. Specifically, the aqueous phase and oil phase may be in an emulsified form and may comprise general ingredients used in emulsion formulations, but are not limited thereto.

In one embodiment, the emulsion formulation may further comprise a thickener. Specifically, the thickener is used to increase the feeling of use by providing an appropriate viscosity when forming the formulation, and a type commonly used in the relevant technical field, for example, triethanolamine, may be used, but the type is not particularly limited.

In one embodiment, the thickener may be contained in an amount of 0.01 to 1% by weight based on the total weight of the emulsion formulation. For example, the thickener may be contained in amount of 0.01% by weight or more, 0.02% by weight or more, 0.03% by weight or more, 0.04% by weight or more, 0.05% by weight or more, 0.06% by weight or more, 0.07% by weight or more, 0.08% by weight or more, 0.09% by weight or more, 0.1% by weight or more, 0.15% by weight or more, 0.2% by weight or more, 0.25% by weight or more, 0.3% by weight or more, 0.35% by weight or more, 0.4% by weight or more, 0.45% by weight or more, 0.5% by weight or more, 1% by weight or less, 0.95% by weight or less, 0.9% by weight or less, 0.85% by weight or less, 0.8% by weight or less, 0.75% by weight or less, 0.7% by weight or less, 0.65% by weight or less, 0.6% by weight or less, or 0.55% by weight or less.

In another aspect, the present invention provides a composition for stabilizing the kojyl methylenedioxycinnamate, wherein the composition comprises a compound comprising an ethylene glycol moiety or a propylene glycol moiety; a surfactant comprising a saccharide moiety; and an antioxidant as an active ingredient, and forms a complex with kojyl methylenedioxycinnamate to stabilize the kojyl methylenedioxycinnamate in an emulsion formulation.

In one embodiment, the kojyl methylenedioxycinnamate may be contained in an amount of 0.1 to 5% by weight based on the total weight of the complex. For example, the kojyl methylenedioxycinnamate may be contained in an amount of 0.1% by weight or more, 0.2% by weight or more, 0.3% by weight or more, 0.5% by weight or more, 0.6% by weight or more, 0.7% by weight or more, 0.8% by weight or more, 0.9% by weight or more, 1% by weight or more, 1.1% by weight or more, 1.2% by weight or more, 1.3% by weight or more, 1.4% by weight or more, 1.5% by weight or more, 1.6% by weight or more, 5% by weight or less, 4.5% by weight or less, 4% by weight or less, 3.5% by weight or less, 3% by weight or less, 2.5% by weight or less, 2.4% by weight or less, 2.3% by weight or less, 2.2% by weight or less, 2.1% by weight or less, 2% by weight or less, 1.9% by weight or less, 1.8% by weight or less, or 1.7% by weight or less.

In one embodiment, the compound comprising an ethylene glycol moiety or a propylene glycol moiety may be contained in an amount of 20 to 60% by weight based on the total weight of the complex. For example, the compound comprising an ethylene glycol moiety or a propylene glycol moiety may be 20% by weight or more, 21% by weight or more, 22% by weight or more, 23% by weight or more, 24% by weight or more, 25% by weight or more, 26% by weight or more, 27% by weight or more, 28% by weight or more, 29% by weight or more, 30% by weight or more, 31% by weight or more, 32% by weight or more, 33% by weight or more, 34% by weight or more, 35% by weight or more, 36% by weight or more, 37% by weight or more, 38% by weight or more, 39% by weight or more, 40% by weight or more, 41% by weight or more, 42% by weight or more, 43% by weight or more, 44% by weight or more, 45% by weight or more, 60% by weight or less, 59% by weight or less, 58% by weight or less, 57% by weight or less, 56% by weight or less, 55% by weight or less, 54% by weight or less, 53% by weight or less, 52% by weight or less, 51% by weight or less, 50% by weight or less, 49% by weight or less, 48% by weight or less, 47% by weight or less, or 46% by weight or less.

In one embodiment, the compound comprising an ethylene glycol moiety or a propylene glycol moiety may have a ratio of carbon and oxygen atoms in the compound of 1.5to 3:1. For example, based on the number of oxygen atoms in the compound as 1, the number of carbon atoms may be 1.5 or more, 1.6 or more, 1.7 or more, 1.8 or more, 1.9 or more, 2 or more, 2.1 or more, 2.2 or more, 2.25 or more, 3 or less, 2.9 or less, 2.8 or less, 2.7 or less, 2.6or less, 2.5 or less, 2.4 or less, or 2.3 or less.

In one embodiment, the compound comprising an ethylene glycol moiety or a propylene glycol moiety may have a distribution coefficient of −2 to 2. For example, the distribution coefficient may be −2 or higher, −1.9 or higher, −1.8 or higher, −1.7 or higher, −1.6 or higher, −1.5 or higher, −1.4 or higher, −1.3 or higher, −1.2 or higher, −1.1 or higher, −1 or higher, −0.9 or higher, −0.8 or higher, −0.7 or higher, −0.6 or higher, −0.5 or higher, −0.4 or higher, −0.3 or higher, −0.2 or higher, −0.1 or higher, 0 or higher, 2 or lower, 1.9 or lower, 1.8 or lower, 1.7 or lower, 1.6 or lower, 1.5 or lower, 1.4 or lower, 1.3 or lower, 1.2 or lower, 1.1 or lower, 1 or lower, 0.9 or lower, 0.8 or lower, 0.7 or lower, 0.6 or lower, 0.5 or lower, 0.4 or lower, 0.3 or lower, 0.2 or lower, or 0.1 or lower.

In one embodiment, the compound comprising an ethylene glycol moiety or a propylene glycol moiety may be at least one selected from the group consisting of dipropylene glycol, diethoxyethyl succinate and polyethylene glycol 400 (PEG400).

In one embodiment, the surfactant comprising a saccharide moiety may be contained in an amount of 30 to 80% by weight based on the total weight of the complex. For example, the surfactant may be present in an amount of 30% by weight or more, 31% by weight or more, 32% by weight or more, 33% by weight or more, 34% by weight or more, 35% by weight or more, 36% by weight or more, 37% by weight or more, 38% by weight or more, 39% by weight or more, 40% by weight or more, 41% by weight or more, 42% by weight or more, 43% by weight or more, 44% by weight or more, 45% by weight or more, 80% by weight or less, 79% by weight or less, 78% by weight or less, 77% by weight or less, 76% by weight or less, 75% by weight or less, 74% by weight or less, 73% by weight or less, 72% by weight or less, 71% by weight or less, 70% by weight or less, 69% by weight or less, 68% by weight or less, 67% by weight or less, 66% by weight or less, 65% by weight or less, 64% by weight or less, 63% by weight or less, 62% by weight or less, 61% by weight or less, 60% by weight or less, 59% by weight or less, 58% by weight or less, 57% by weight or less, 56% by weight or less, 55% by weight or less, 54% by weight or less, 53% by weight or less, 52% by weight or less, 51% by weight or less, 50% by weight or less, 49% by weight or less, 48% by weight or less, 47% by weight or less, or 46% by weight or less.

In one embodiment, the surfactant comprising a saccharide moiety may have an HLB (Hydrophile-Lipophile Balance) value of 15.5 to 18.5. For example, the HLB value may be 15.5 or higher, 15.6 or higher, 15.7 or higher, 15.8 or higher, 15.9 or higher, 16 or higher, 16.1 or higher, 16.2 or higher, 16.3 or higher, 16.4 or higher, 16.5 or higher, 16.6 or higher, 16.7 or higher, 16.8 or higher, 16.9 or higher, 17 or higher, 18.5 or lower, 18.4 or lower, 18.3 or lower, 18.2 or lower, 18.1 or lower, 18 or lower, 17.9 or lower, 17.8 or lower, 17.7 or lower, 17.6 or lower, 17.5 or lower, 17.4 or lower, 17.3 or lower, 17.2 or lower, or 17.1 or lower.

In one embodiment, the surfactant comprising a saccharide moiety may be at least one selected from the group consisting of polysorbate 20 and sucrose laurate.

In one embodiment, the antioxidant may be contained in an amount of 0.1 to 5% by weight based on the total weight of the complex. For example, the antioxidant may be present in an amount of 0.1% by weight or more, 0.2% by weight or more, 0.3% by weight or more, 0.5% by weight or more, 0.6% by weight or more, 0.7% by weight or more, 0.8% by weight or more, 0.9% by weight or more, 1% by weight or more, 1.1% by weight or more, 1.2% by weight or more, 1.3% by weight or more, 1.4% by weight or more, 1.5% by weight or more, 1.6% by weight or more, 5% by weight or less, 4.5% by weight or less, 4% by weight or less, 3.5% by weight or less, 3% by weight or less, 2.5% by weight or less, 2.4% by weight or less, 2.3% by weight or less, 2.2% by weight or less, 2.1% by weight or less, 2% by weight or less, 1.9% by weight or less, 1.8% by weight or less, or 1.7% by weight or less.

In one embodiment, the antioxidant may be at least one selected from the group consisting of tocopherol and ferulic acid.

In another aspect, the present invention provides a method for stabilizing kojyl methylenedioxycinnamate, comprising: mixing kojyl methylenedioxycinnamate with a compound comprising an ethylene glycol moiety or a propylene glycol moiety; a surfactant comprising a saccharide moiety; and an antioxidant to form a complex and to stabilize the kojyl methylenedioxycinnamate in an emulsion formulation.

In another aspect, the present invention provides a method for manufacturing the complex, comprising: (a) adding kojyl methylenedioxycinnamate, a compound comprising an ethylene glycol moiety or a propylene glycol moiety, a surfactant comprising a saccharide moiety, and an antioxidant; and (b) homomixing the result of the (a).

The kojyl methylenedioxycinnamate has a plate-like structure, so the plate-like structures exhibit a stacking phenomenon, which causes precipitation from the formulation. Accordingly, when strong physical force is applied to kojyl methylenedioxycinnamate, the stacking phenomenon can be prevented. Therefore, a homomixing step in which a strong physical force is applied rather than a general stirring method is necessary in a solution containing kojyl methylenedioxycinnamate.

In one embodiment, the homomixing in the (b) may be performed at 3,000 to 10,000 rpm for 30 to 400 minutes. For example, the homomixing may be performed at a speed of 3,000 rpm or more, 3,500 rpm or more, 4,000 rpm or more, 4,500 rpm or more, 5,000 rpm or more, 5,500 rpm or more, 6,000 rpm or more, 6,100 rpm or more, 6,200 rpm or more, 6,300 rpm or more, 6,400 rpm or more, 6,500 rpm or more, 10,000 rpm or less, 9,500 rpm or less, 9,000 rpm or less, 8,500 rpm or less, 8,000 rpm or less, 7,500 rpm or less, 7,000 rpm or less, 6,900 rpm or less, 6,800 rpm or less, 6,700 rpm or less, or 6,600 rpm or less. Also, for example, the homomixing may be performed for 30 minutes or more, 40 minutes or more, 50 minutes or more, 60 minutes or more, 70 minutes or more, 80 minutes or more, 90 minutes or more, 100 minutes or more, 110 minutes or more, 120 minutes or more, 130 minutes or more, 140 minutes or more, 150 minutes or more, 160 minutes or more, 170 minutes or more, 180 minutes or more, 190 minutes or more, 200 minutes or more, 400 minutes or less, 390 minutes or less, 380 minutes or less, 370 minutes or less, 360 minutes or less, 350 minutes or less, 340 minutes or less, 330 minutes or less, 320 minutes or less, 310 minutes or less, 300 minutes or less, 290 minutes or less, 280 minutes or less, 270 minutes or less, 260 minutes or less, 250 minutes or less, 240 minutes or less, 230 minutes or less, 220 minutes or less, or 210 minutes or less.

In one embodiment, the kojyl methylenedioxycinnamate may be added in an amount of 0.1 to 5% by weight based on the total weight of the complex. For example, the kojyl methylenedioxycinnamate may be contained in an amount of 0.1% by weight or more, 0.2% by weight or more, 0.3% by weight or more, 0.5% by weight or more, 0.6% by weight or more, 0.7% by weight or more, 0.8% by weight or more, 0.9% by weight or more, 1% by weight or more, 1.1% by weight or more, 1.2% by weight or more, 1.3% by weight or more, 1.4% by weight or more, 1.5% by weight or more, 1.6% by weight or more, 5% by weight or less, 4.5% by weight or less, 4% by weight or less, 3.5% by weight or less, 3% by weight or less, 2.5% by weight or less, 2.4% by weight or less, 2.3% by weight or less, 2.2% by weight or less, 2.1% by weight or less, 2% by weight or less, 1.9% by weight or less, 1.8% by weight or less, or 1.7% by weight or less.

In one embodiment, the compound comprising an ethylene glycol moiety or a propylene glycol moiety may be added in an amount of 20 to 60% by weight based on the total weight of the complex. For example, the compound comprising an ethylene glycol moiety or a propylene glycol moiety may be 20% by weight or more, 21% by weight or more, 22% by weight or more, 23% by weight or more, 24% by weight or more, 25% by weight or more, 26% by weight or more, 27% by weight or more, 28% by weight or more, 29% by weight or more, 30% by weight or more, 31% by weight or more, 32% by weight or more, 33% by weight or more, 34% by weight or more, 35% by weight or more, 36% by weight or more, 37% by weight or more, 38% by weight or more, 39% by weight or more, 40% by weight or more, 41% by weight or more, 42% by weight or more, 43% by weight or more, 44% by weight or more, 45% by weight or more, 60% by weight or less, 59% by weight or less, 58% by weight or less, 57% by weight or less, 56% by weight or less, 55% by weight or less, 54% by weight or less, 53% by weight or less, 52% by weight or less, 51% by weight or less, 50% by weight or less, 49% by weight or less, 48% by weight or less, 47% by weight or less, or 46% by weight or less.

In one embodiment, the compound comprising an ethylene glycol moiety or a propylene glycol moiety may have a ratio of carbon and oxygen atoms in the compound of 1.5 to 3:1. For example, based on the number of oxygen atoms in the compound as 1, the number of carbon atoms may be 1.5 or more, 1.6 or more, 1.7 or more, 1.8 or more, 1.9 or more, 2 or more, 2.1 or more, 2.2 or more, 2.25 or more, 3 or less, 2.9 or less, 2.8 or less, 2.7 or less, 2.6 or less, 2.5 or less, 2.4 or less, or 2.3 or less.

In one embodiment, the compound comprising an ethylene glycol moiety or a propylene glycol moiety may have a distribution coefficient of −2 to 2. For example, the distribution coefficient may be −2 or higher, −1.9 or higher, −1.8 or higher, −1.7 or higher, −1.6 or higher, −1.5 or higher, −1.4 or higher, −1.3 or higher, −1.2 or higher, −1.1 or higher, −1 or higher, −0.9 or higher, −0.8 or higher, −0.7 or higher, −0.6 or higher, −0.5 or higher, −0.4 or higher, −0.3 or higher, −0.2 or higher, −0.1 or higher, 0 or higher, 2 or lower, 1.9 or lower, 1.8 or lower, 1.7 or lower, 1.6 or lower, 1.5 or lower, 1.4 or lower, 1.3 or lower, 1.2 or lower, 1.1 or lower, 1 or lower, 0.9 or lower, 0.8 or lower, 0.7 or lower, 0.6 or lower, 0.5 or lower, 0.4 or lower, 0.3 or lower, 0.2 or lower, or 0.1 or lower.

In one embodiment, the compound comprising an ethylene glycol moiety or a propylene glycol moiety may be at least one selected from the group consisting of dipropylene glycol, diethoxyethyl succinate and polyethylene glycol 400 (PEG400).

In one embodiment, the surfactant comprising a saccharide moiety may be added in an amount of 30 to 80% by weight based on the total weight of the complex. For example, the surfactant may be present in an amount of 30% by weight or more, 31% by weight or more, 32% by weight or more, 33% by weight or more, 34% by weight or more, 35% by weight or more, 36% by weight or more, 37% by weight or more, 38% by weight or more, 39% by weight or more, 40% by weight or more, 41% by weight or more, 42% by weight or more, 43% by weight or more, 44% by weight or more, 45% by weight or more, 80% by weight or less, 79% by weight or less, 78% by weight or less, 77% by weight or less, 76% by weight or less, 75% by weight or less, 74% by weight or less, 73% by weight or less, 72% by weight or less, 71% by weight or less, 70% by weight or less, 69% by weight or less, 68% by weight or less, 67% by weight or less, 66% by weight or less, 65% by weight or less, 64% by weight or less, 63% by weight or less, 62% by weight or less, 61% by weight or less, 60% by weight or less, 59% by weight or less, 58% by weight or less, 57% by weight or less, 56% by weight or less, 55% by weight or less, 54% by weight or less, 53% by weight or less, 52% by weight or less, 51% by weight or less, 50% by weight or less, 49% by weight or less, 48% by weight or less, 47% by weight or less, or 46% by weight or less.

In one embodiment, the surfactant comprising a saccharide moiety may have an HLB (Hydrophile-Lipophile Balance) value of 15.5 to 18.5. For example, the HLB value may be 15.5 or higher, 15.6 or higher, 15.7 or higher, 15.8 or higher, 15.9 or higher, 16 or higher, 16.1 or higher, 16.2 or higher, 16.3 or higher, 16.4 or higher, 16.5 or higher, 16.6 or higher, 16.7 or higher, 16.8 or higher, 16.9 or higher, 17 or higher, 18.5 or lower, 18.4 or lower, 18.3 or lower, 18.2 or lower, 18.1 or lower, 18 or lower, 17.9 or lower, 17.8 or lower, 17.7 or lower, 17.6 or lower, 17.5 or lower, 17.4 or lower, 17.3 or lower, 17.2 or lower, or 17.1 or lower.

In one embodiment, the surfactant comprising a saccharide moiety may be at least one selected from the group consisting of polysorbate 20 and sucrose laurate.

In one embodiment, the antioxidant may be added in an amount of 0.1 to 5% by weight based on the total weight of the complex. For example, the antioxidant may be present in an amount of 0.1% by weight or more, 0.2% by weight or more, 0.3% by weight or more, 0.5% by weight or more, 0.6% by weight or more, 0.7% by weight or more, 0.8% by weight or more, 0.9% by weight or more, 1% by weight or more, 1.1% by weight or more, 1.2% by weight or more, 1.3% by weight or more, 1.4% by weight or more, 1.5% by weight or more, 1.6% by weight or more, 5% by weight or less, 4.5% by weight or less, 4% by weight or less, 3.5% by weight or less, 3% by weight or less, 2.5% by weight or less, 2.4% by weight or less, 2.3% by weight or less, 2.2% by weight or less, 2.1% by weight or less, 2% by weight or less, 1.9% by weight or less, 1.8% by weight or less, or 1.7% by weight or less.

In one embodiment, the antioxidant may be at least one selected from the group consisting of tocopherol and ferulic acid.

In one embodiment, the method may be performed at 60 to 80° C. For example, it may be performed at 60° C. or higher, 61° C. or higher, 62° C. or higher, 63° C. or higher, 64° C. or higher, 65° C. or higher, 66° C. or higher, 67° C. or higher, 68° C. or higher, 69° C. or higher, 70° C. or higher, 80° C. or lower, 79° C. or lower, 78° C. or lower, 77° C. or lower, 76° C. or lower, 75° C. or lower, 74° C. or lower, 73° C. or lower, 72° C. or lower, or 71° C. or lower.

In another aspect, the present invention provides a method for manufacturing the emulsion formulation, comprising: (a) preparing an emulsion by preparing an aqueous phase and an oil phase; (b) independently of the (a), adding kojyl methylenedioxycinnamate, a compound comprising an ethylene glycol moiety or a propylene glycol moiety, a surfactant comprising a saccharide moiety, and an antioxidant; (c) homomixing the result of the (b); and (d) adding the result of the (c) to the oil phase of the (a).

In one embodiment, the homomixing in the (c) may be performed at 3,000 to 10,000 rpm for 30 to 400 minutes. For example, the homomixing may be performed at a speed of 3,000 rpm or more, 3,500 rpm or more, 4,000 rpm or more, 4,500 rpm or more, 5,000 rpm or more, 5,500 rpm or more, 6,000 rpm or more, 6,100 rpm or more, 6,200 rpm or more, 6,300 rpm or more, 6,400 rpm or more, 6,500 rpm or more, 10,000 rpm or less, 9,500 rpm or less, 9,000 rpm or less, 8,500 rpm or less, 8,000 rpm or less, 7,500 rpm or less, 7,000 rpm or less, 6,900 rpm or less, 6,800 rpm or less, 6,700 rpm or less, or 6,600 rpm or less. Also, for example, the homomixing may be performed for 30 minutes or more, 40 minutes or more, 50 minutes or more, 60 minutes or more, 70 minutes or more, 80 minutes or more, 90 minutes or more, 100 minutes or more, 110 minutes or more, 120 minutes or more, 130 minutes or more, 140 minutes or more, 150 minutes or more, 160 minutes or more, 170 minutes or more, 180 minutes or more, 190 minutes or more, 200 minutes or more, 400 minutes or less, 390 minutes or less, 380 minutes or less, 370 minutes or less, 360 minutes or less, 350 minutes or less, 340 minutes or less, 330 minutes or less, 320 minutes or less, 310 minutes or less, 300 minutes or less, 290 minutes or less, 280 minutes or less, 270 minutes or less, 260 minutes or less, 250 minutes or less, 240 minutes or less, 230 minutes or less, 220 minutes or less, or 210 minutes or less.

In one embodiment, in the (d), the result of the (c) may be added in an amount of 1 to 5% by weight based on the total weight of the emulsion formulation. For example, the complex may be present in amount of 1% by weight or more, 1.1% by weight or more, 1.2% by weight or more, 1.3% by weight or more, 1.4% by weight or more, 1.5% by weight or more, 1.6% by weight or more, 1.7% by weight or more, 1.8% by weight or more, 1.9% by weight or more, 2% by weight or more, 2.1% by weight or more, 2.2% by weight or more, 2.3% by weight or more, 2.4% by weight or more, 2.5% by weight or more, 2.6% by weight or more, 2.7% by weight or more, 2.8% by weight or more, 2.9% by weight or more, 3% by weight or more, 5% by weight or less, 4.9% by weight or less, 4.8% by weight or less, 4.7% by weight or less, 4.6% by weight or less, 4.5% by weight or less, 4.4% by weight or less, 4.3% by weight or less, 4.2% by weight or less, 4.1% by weight or less, 4% by weight or less, 3.9% by weight or less, 3.8% by weight or less, 3.7% by weight or less, 3.6% by weight or less, 3.5% by weight or less, 3.4% by weight or less, 3.3% by weight or less, 3.2% by weight or less, or 3.1% by weight or less.

In one embodiment, the kojyl methylenedioxycinnamate may be added in an amount of 0.1 to 5% by weight based on the total weight of the result of the (c). For example, the kojyl methylenedioxycinnamate may be present in amount of 0.1% by weight or more, 0.2% by weight or more, 0.3% by weight or more, 0.5% by weight or more, 0.6% by weight or more, 0.7% by weight or more, 0.8% by weight or more, 0.9% by weight or more, 1% by weight or more, 1.1% by weight or more, 1.2% by weight or more, 1.3% by weight or more, 1.4% by weight or more, 1.5% by weight or more, 1.6% by weight or more, 5% by weight or less, 4.5% by weight or less, 4% by weight or less, 3.5% by weight or less, 3% by weight or less, 2.5% by weight or less, 2.4% by weight or less, 2.3% by weight or less, 2.2% by weight or less, 2.1% by weight or less, 2% by weight or less, 1.9% by weight or less, 1.8% by weight or less, or 1.7% by weight or less.

In one embodiment, the compound comprising an ethylene glycol moiety or a propylene glycol moiety may be added in an amount of more than 30 to 60% by weight based on the total weight of the result of the (c). For example, the compound comprising an ethylene glycol moiety or a propylene glycol moiety may be present in amount of 20% by weight or more, 21% by weight or more, 22% by weight or more, 23% by weight or more, 24% by weight or more, 25% by weight or more, 26% by weight or more, 27% by weight or more, 28% by weight or more, 29% by weight or more, 30% by weight or more, 31% by weight or more, 32% by weight or more, 33% by weight or more, 34% by weight or more, 35% by weight or more, 36% by weight or more, 37% by weight or more, 38% by weight or more, 39% by weight or more, 40% by weight or more, 41% by weight or more, 42% by weight or more, 43% by weight or more, 44% by weight or more, 45% by weight or more, 60% by weight or less, 59% by weight or less, 58% by weight or less, 57% by weight or less, 56% by weight or less, 55% by weight or less, 54% by weight or less, 53% by weight or less, 52% by weight or less, 51% by weight or less, 50% by weight or less, 49% by weight or less, 48% by weight or less, 47% by weight or less, or 46% by weight or less.

In one embodiment, the compound comprising an ethylene glycol moiety or a propylene glycol moiety may have a ratio of carbon and oxygen atoms in the compound of 1.5 to 3:1. For example, based on the number of oxygen atoms in the compound as 1, the number of carbon atoms may be 1.5 or more, 1.6 or more, 1.7 or more, 1.8 or more, 1.9 or more, 2 or more, 2.1 or more, 2.2 or more, 2.25 or more, 3 or less, 2.9 or less, 2.8 or less, 2.7 or less, 2.6 or less, 2.5 or less, 2.4 or less, or 2.3 or less.

In one embodiment, the compound comprising an ethylene glycol moiety or a propylene glycol moiety may have a distribution coefficient of −2 to 2. For example, the distribution coefficient may be −2 or higher, −1.9 or higher, −1.8 or higher, −1.7 or higher, −1.6 or higher, −1.5 or higher, −1.4 or higher, −1.3 or higher, −1.2 or higher, −1.1 or higher, −1 or higher, −0.9 or higher, −0.8 or higher, −0.7 or higher, −0.6 or higher, −0.5 or higher, −0.4 or higher, −0.3 or higher, −0.2 or higher, −0.1 or higher, 0 or higher, 2 or lower, 1.9 or lower, 1.8 or lower, 1.7 or lower, 1.6 or lower, 1.5 or lower, 1.4 or lower, 1.3 or lower, 1.2 or lower, 1.1 or lower, 1 or lower, 0.9 or lower, 0.8 or lower, 0.7 or lower, 0.6 or lower, 0.5 or lower, 0.4 or lower, 0.3 or lower, 0.2 or lower, or 0.1.

In one embodiment, the compound comprising an ethylene glycol moiety or a propylene glycol moiety may be at least one selected from the group consisting of dipropylene glycol, diethoxyethyl succinate and polyethylene glycol 400 (PEG400).

In one embodiment, the surfactant comprising a saccharide moiety may be added in an amount of 30 to 80% by weight based on the total weight of the result of the (c). For example, the surfactant may be present in an amount of 30% by weight or more, 31% by weight or more, 32% by weight or more, 33% by weight or more, 34% by weight or more, 35% by weight or more, 36% by weight or more, 37% by weight or more, 38% by weight or more, 39% by weight or more, 40% by weight or more, 41% by weight or more, 42% by weight or more, 43% by weight or more, 44% by weight or more, 45% by weight or more, 80% by weight or less, 79% by weight or less, 78% by weight or less, 77% by weight or less, 76% by weight or less, 75% by weight or less, 74% by weight or less, 73% by weight or less, 72% by weight or less, 71% by weight or less, 70% by weight or less, 69% by weight or less, 68% by weight or less, 67% by weight or less, 66% by weight or less, 65% by weight or less, 64% by weight or less, 63% by weight or less, 62% by weight or less, 61% by weight or less, 60% by weight or less, 59% by weight or less, 58% by weight or less, 57% by weight or less, 56% by weight or less, 55% by weight or less, 54% by weight or less, 53% by weight or less, 52% by weight or less, 51% by weight or less, 50% by weight or less, 49% by weight or less, 48% by weight or less, 47% by weight or less, or 46% by weight or less.

In one embodiment, the surfactant comprising a saccharide moiety may have an HLB (Hydrophile-Lipophile Balance) value of 15.5 to 18.5. For example, the HLB value may be 15.5 or higher, 15.6 or higher, 15.7 or higher, 15.8 or higher, 15.9 or higher, 16 or higher, 16.1 or higher, 16.2 or higher, 16.3 or higher, 16.4 or higher, 16.5 or higher, 16.6 or higher, 16.7 or higher, 16.8 or higher, 16.9 or higher, 17 or higher, 18.5 or lower, 18.4 or lower, 18.3 or lower, 18.2 or lower, 18.1 or lower, 18 or lower, 17.9 or lower, 17.8 or lower, 17.7 or lower, 17.6 or lower, 17.5 or lower, 17.4 or lower, 17.3 or lower, 17.2 or lower, or 17.1 or lower.

In one embodiment, the surfactant comprising a saccharide moiety may be at least one selected from the group consisting of polysorbate 20 and sucrose laurate.

In one embodiment, the antioxidant may be added in an amount of 0.1 to 5% by weight based on the total weight of the result of the (c). For example, the antioxidant may be present in an amount of 0.1% by weight or more, 0.2% by weight or more, 0.3% by weight or more, 0.5% by weight or more, 0.6% by weight or more, 0.7% by weight or more, 0.8% by weight or more, 0.9% by weight or more, 1% by weight or more, 1.1% by weight or more, 1.2% by weight or more, 1.3% by weight or more, 1.4% by weight or more, 1.5% by weight or more, 1.6% by weight or more, 5% by weight or less, 4.5% by weight or less, 4% by weight or less, 3.5% by weight or less, 3% by weight or less, 2.5% by weight or less, 2.4% by weight or less, 2.3% by weight or less, 2.2% by weight or less, 2.1% by weight or less, 2% by weight or less, 1.9% by weight or less, 1.8% by weight or less, or 1.7% by weight or less.

In one embodiment, the antioxidant may be at least one selected from the group consisting of tocopherol and ferulic acid.

In one embodiment, the method may be performed at 60 to 80° C. For example, it may be performed at 60° C. or higher, 61° C. or higher, 62° C. or higher, 63° C. or higher, 64° C. or higher, 65° C. or higher, 66° C. or higher, 67° C. or higher, 68° C. or higher, 69° C. or higher, 70° C. or higher, 80° C. or lower, 79° C. or lower, 78° C. or lower, 77° C. or lower, 76° C. or lower, 75° C. or lower, 74° C. or lower, 73° C. or lower, 72° C. or lower, or 71° C. or lower.

Hereinafter, the contents of the present invention are described in more detail through examples. However, these examples are only presented to understand the contents of the present invention, and the scope of the present invention is not limited to these examples. Modifications, substitutions, and insertions commonly known in the art can be performed, and these are also included within the scope of the present invention.

### EXAMPLES

### 1. Preparation of Complex

A complex was prepared according to the composition shown in Table 1 below. Each component was placed in a container, and a complex was prepared by each stirring method at 70° C. Complexes 1 to 3 and Complexes 6 to 12 were homomixed at 8000 rpm for 120 minutes using a homomixer (PRIMIX, T.K. Homomixture MARK II), and Complex 4 was stirred for 120 minutes using a stirrer by putting a magnetic bar into the solution. Complex 5 was azimixed at 300 rpm for 120 minutes using an azimixer.

TABLE 1

| | | Complex No. | | | | | | | | | | | |
|---|---|---|---|---|---|---|---|---|---|---|---|---|---|
| Classification | | 1 | 2 | 3 | 4 | 5 | 6 | 7 | 8 | 9 | 10 | 11 | 12 |
| Ingredient | Kojyl | 1.6 | 1.6 | 1.6 | 1.6 | 1.6 | 1.6 | 1.6 | 1.6 | 1.6 | 1.6 | 1.6 | 1.6 |
| | methylenedioxycinnamate | | | | | | | | | | | | |
| | Dipropylene glycol | 32.3 | | 32.3 | 32.3 | 32.3 | 33.9 | | 32.3 | 32.3 | 10 | 80 | |
| | Diethoxyethyl succinate | | 32.3 | | | | | | | | | | |
| | 1,3-BG | | | | | | | 32.3 | | | | | |
| | Diethylene glycol | | | | | | | | | | | | 32.3 |
| | lauryl ether | | | | | | | | | | | | |
| | Polysorbate 20 | 64.5 | 64.5 | 64.5 | 64.5 | 64.5 | 64.5 | 64.5 | | | 88.4 | 18.4 | 64.5 |
| | Polysorbate 60 | | | | | | | | 64.5 | | | | |
| | Laureth-23 | | | | | | | | | 64.5 | | | |
| | MEL | | | | | | | | | | | | |
| | Delta tocopherol | 1.6 | 1.6 | | 1.6 | 1.6 | | 1.6 | 1.6 | 1.6 | | | 1.6 |
| | Ferulic acid | | | 1.6 | | | | | | | | | |
| Stirring | Magnetic stirring | — | — | — | ◯ | — | — | — | — | — | — | — | — |
| method | Azi mixing | — | — | — | — | ◯ | — | — | — | — | — | — | — |
| | Homo mixing | ◯ | ◯ | ◯ | — | — | ◯ | ◯ | ◯ | ◯ | ◯ | ◯ | ◯ |

Among the above ingredients, the logP values of dipropylene glycol, diethoxyethyl 5 succinate. 1.3-BG. and diethylene glycol lauryl ether were shown in [Table 2]. and the HLB values of polysorbate 20. polysorbate 60 and laureth-23 were shown in [Table 3].

TABLE 2

| | Whether it contains a | | |
|---|---|---|---|
| | moiety of ethylene glycol | | |
| Ingredient | or propylene glycol | C:O | logP |
| Dipropylene glycol | ◯ | 6:3 | −0.31 |
| Diethoxyethyl succinate | ◯ | 12:6 | 0.25 |
| 1,3-BG | X | — | — |
| Diethylene glycol lauryl ether | ◯ | 16:3 | 4 |

TABLE 3

| | Whether it contains a | |
|---|---|---|
| Ingredient | moiety of saccharide | HLB |
| Polysorbate 20 | ◯ | 17 |
| Polysorbate 60 | ◯ | 15 |
| Laureth-23 | X | — |

### 2. Preparation of Emulsion Composition

An emulsion composition was prepared as shown in [Table 4] below. The aqueous phase was prepared by adding the components of the aqueous phase at 70° C., and the oil phase was prepared by adding the components of the oil phase at 70° C. in a separate container. The prepared oil phase was added to the previously prepared aqueous phase, and stirred at 8000 rpm for 5 minutes using a homomixer for primary emulsification, and triethanolamine was added to the resulting product to thicken it. Afterwards, each of Complexes 1 to 12 was added and named as shown in [Table 5]. Comparative Example 10 was prepared by adding 10 components of the same composition as Complex 1, respectively, without preparing a complex.

TABLE 4

| Classification | Ingredient | Weight % |
|---|---|---|
| Aqueous phase | Purified water | Up to 100 |
| | Glycerin | 8 |
| | Butylene glycol | 4 |
| | Carbomer | 0.1 |
| Oil phase | Caprylic Capric Triglyceride | 8 |
| | Hydrogenated Plidecene | 5 |
| | Cetearyl glucoside | 1.5 |
| | Sorbitan stearate | 0.4 |
| | Cetearyl alcohol | 1 |
| | Antiseptic | Appropriate amount |
| | Flavor | Appropriate amount |
| Others | Triethanolamine | 0.1 |
| | Complex | 3 |

TABLE 5

| Emulsion composition nomenclature | Complex types included |
|---|---|
| Example 1 | Complex 1 |
| Example 2 | Complex 2 |
| Example 3 | Complex 3 |
| Comparative Example 1 | Complex 4 |
| Comparative Example 2 | Complex 5 |
| Comparative Example 3 | Complex 6 |
| Comparative Example 4 | Complex 7 |
| Comparative Example 5 | Complex 8 |
| Comparative Example 6 | Complex 9 |
| Comparative Example 7 | Complex 10 |
| Comparative Example 8 | Complex 11 |
| Comparative Example 9 | Complex 12 |
| Comparative Example 10 | Adding each complex component |
| | without preparing a complex |

### 3. Evaluation of Formulation Stability of Emulsion Composition

### (1) Precipitation Evaluation of Kojyl Methylenedioxycinnamate

The prepared emulsion composition was manufactured at room temperature and then stored in a constant temperature chamber at room temperature and 40° C. to visually observe whether crystals were formed over time, and the results are shown in [Table 6] below. X indicates that crystals were observed, Δ indicates that very fine crystals were observed, and ◯ indicates that no crystals appeared.

As a result, no crystals were observed in Examples 1 to 3, but crystals were observed in Comparative Examples 1 to 10.

TABLE 6

| | | | | | Compar- | Compar- | Compar- | Compar- | Compar- | Compar- | Compar- | Compar- | Compar- |
|---|---|---|---|---|---|---|---|---|---|---|---|---|---|
| | | | | | ative | ative | ative | ative | ative | ative | ative | ative | ative |
| | | Ex. 1 | Ex. 2 | Ex. 3 | Ex. 1 | Ex. 2 | Ex. 4 | Ex. 5 | Ex. 6 | Ex. 7 | Ex. 8 | Ex. 9 | Ex. 10 |
| Immediately | | ◯ | ◯ | ◯ | X | Δ | X | Δ | X | Δ | Δ | X | X |
| after | | | | | | | | | | | | | |
| preparation | | | | | | | | | | | | | |
| 1 | day | ◯ | ◯ | ◯ | X | X | X | Δ | X | X | Δ | X | X |
| 1 | week | ◯ | ◯ | ◯ | X | X | X | X | X | X | Δ | X | X |
| 2 | weeks | ◯ | ◯ | ◯ | X | X | X | X | X | X | X | X | X |
| 10 | weeks | ◯ | ◯ | ◯ | X | X | X | X | X | X | X | X | X |

### (2) Potency Evaluation of Kojyl Methylenedioxycinnamate

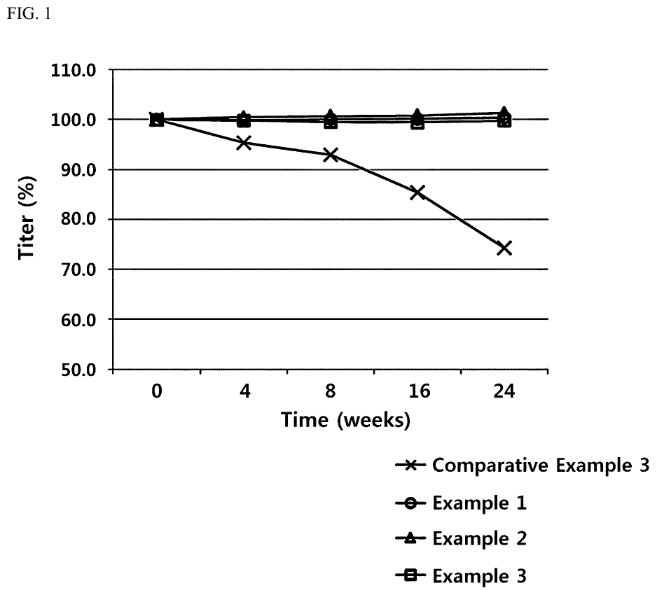

2.0 g of each samples of the prepared emulsion composition prepared immediately after preparation, 4 weeks later, 8 weeks later, 16 weeks later, and 24 weeks later were precisely weighed. Then, 20 mL of dimethylformamide was added and dispersed by ultrasonic waves, and then methanol was added to make 100 mL to prepare a sample solution. Exactly 10 mL of this solution was taken and dimethylformamide/methanol mixture (2:8) was added to make 100 mL, which was used as the standard solution. 10 μL of each sample solution and standard solution were passed through a reversed-phase column to a mixed solution of acetonitrile and water at a flow rate of 1 mL per minute, and the results were tested according to the liquid chromatography method under the conditions of analysis at 330 nm using an ultraviolet absorbance spectrophotometer. The results are shown in FIG. 1.

As a result, in the case of Comparative Example 3, which did not contain antioxidants, it was confirmed that the content of kojyl methylenedioxycinnamate began to decrease over time immediately after preparation and the content decreased by about 27% 24 weeks after preparation. In contrast, in the case of Examples 1 to 3, it was confirmed that the content of kojyl methylenedioxycinnamate was maintained until 24 weeks after preparation.

### 4. Evaluation of Skin Absorption of the Emulsion Formulation

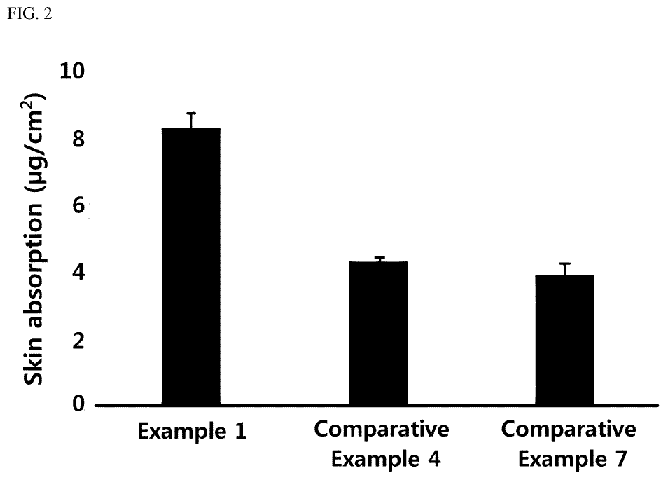

The degree of skin absorption was evaluated for Example 1, and Comparative Examples 4 and 7. A Strat M membrane (Merck Millipore, USA) similar to human skin was fixed between donor and receptor phases, and the receptor phase was filled with PBS and the temperature was maintained at 32±1° C. through a constant temperature water bath. After loading the experimental group on the donor, the receptor phase was collected at regular time intervals for 24 hours, and the Strat M membrane was separated, washed, and extracted. This was quantified through HPLC analysis. As shown in FIG. 2, in the cased of Example 1, the skin absorption amount was about 8 μg/cm², while in the case of Comparative Examples 4 and 7, the skin absorption amount was less than 5 μg/cm².

What is claimed is:

1. A complex comprising kojyl methylenedioxycinnamate; a compound comprising an ethylene glycol moiety or a propylene glycol moiety; a surfactant comprising a saccharide moiety; and an antioxidant.

2. The complex according to claim 1, wherein the kojyl methylenedioxycinnamate is contained in an amount of 0.1 to 5% by weight based on the total weight of the complex.

3. The complex according to claim 1, wherein the compound comprising an ethylene glycol moiety or a propylene glycol moiety comprises two or more ethylene glycol moieties or propylene glycol moieties.

4. The complex according to claim 1, wherein the compound comprising an ethylene glycol moiety or a propylene glycol moiety is contained in an amount of 20 to 60% by weight based on the total weight of the complex.

5. The complex according to claim 1, wherein the compound comprising an ethylene glycol moiety or a propylene glycol moiety has a ratio of carbon and oxygen atoms in the compound of 1.5-3:1, and/or
wherein the compound comprising an ethylene glycol moiety or a propylene glycol moiety has a distribution coefficient of −2 to 2, and/or
wherein the compound comprising an ethylene glycol moiety or a propylene glycol moiety is at least one selected from the group consisting of dipropylene glycol, diethoxyethyl succinate and polyethylene glycol 400.

6. The complex according to claim 1, wherein the surfactant comprising a saccharide moiety is contained in an amount of 30 to 80% by weight based on the total weight of the complex.

7. The complex according to claim 1, wherein the surfactant comprising a saccharide moiety has an HLB value of 15.5 to 18.5, and/or
wherein the surfactant comprising a saccharide moiety is at least one selected from the group consisting of polysorbate 20 and sucrose laurate.

8. The complex according to claim 1, wherein the antioxidant is contained in an amount of 0.1 to 5% by weight based on the total weight of the complex, and/or
wherein the antioxidant is at least one selected from the group consisting of tocopherol and ferulic acid.

9. An emulsion formulation comprising the complex of claim 1.

10. The emulsion formulation according to claim 9, wherein the complex is contained in an amount of 1 to 10% by weight based on the total weight of the emulsion formulation.

11. A method for stabilizing kojyl methylenedioxycinnamate in an emulsion formulation, comprising mixing kojyl methylenedioxycinnamate with a compound comprising an ethylene glycol moiety or a propylene glycol moiety; a surfactant comprising a saccharide moiety; and an antioxidant;
wherein the compound comprising an ethylene glycol moiety or a propylene glycol moiety; the surfactant comprising a saccharide moiety; and the antioxidant forms a complex with kojyl methylenedioxycinnamate.

12. The method according to claim 11, wherein the compound comprising an ethylene glycol moiety or a propylene glycol moiety comprises two or more ethylene glycol moieties or propylene glycol moieties.

13. The method according to claim 11, wherein the compound comprising an ethylene glycol moiety or a propylene glycol moiety is contained in an amount of 20 to 60% by weight based on the total weight of the complex.

14. The method according to claim 11, wherein the surfactant comprising a saccharide moiety is contained in an amount of 30 to 80% by weight based on the total weight of the complex.

15. The method according to claim 11, wherein the antioxidant is contained in an amount of 0.1 to 5% by weight based on the total weight of the complex.

16. A method for manufacturing the complex of claim 1, comprising:
(a) adding kojyl methylenedioxycinnamate, a compound comprising an ethylene glycol moiety or a propylene glycol moiety, a surfactant comprising a saccharide moiety, and an antioxidant; and
(b) homomixing the result of the (a).

17. The method according to claim 16, wherein the homomixing in the (b) is performed at 3000 to 10000 rpm for 30 to 400 minutes, and/or
wherein the method is performed at 60 to 80° C.

18. A method for manufacturing the emulsion formulation of claim 9, comprising:
(a) preparing an emulsion by preparing an aqueous phase and an oil phase;
(b) independently of the (a), adding kojyl methylenedioxycinnamate, a compound comprising an ethylene glycol moiety or a propylene glycol moiety, a surfactant comprising a saccharide moiety, and an antioxidant;
(c) homomixing the result of the (b); and
(d) adding the result of the (c) to the oil phase of the (a).

19. The method according to claim 18, wherein in the (d), the result of the (c) is added in an amount of 1 to 10% by weight based on the total weight of the emulsion formulation.

20. The method according to claim 18, wherein the (a) further comprises adding a thickener after preparing the emulsion.
